SEQUENCE LISTING (1 sequence)

SEQ ID NO: 1 - protein, 26 aa, Artificial Sequence
  c-MET peptide
AGSCYCSGPPRFECWCYETEGTGGGK

(19) United States
(12) Patent Application Publication
(10) Pub. No.: US 2019/0151483 A1
(43) Pub. Date: May 23, 2019

(54) METHODS OF MAKING 18F-LABELED PRECURSORS AND PEPTIDES, LABELED C-MET BINDING PEPTIDES, AND METHODS OF USE THEREOF

(71) Applicant: The United States of America, as Represented by the Secretary, Department of Health and Human Serv, Bethesda, MD (US)

(72) Inventors: Falguni Bhattacharyya, Clarksburg, MD (US); Rolf Swenson, Silver Spring, MD (US); Elaine Marie Jagoda, Derwood, MD (US)

(21) Appl. No.: 16/320,645

(22) PCT Filed: Jul. 25, 2017
(86) PCT No.: PCT/US2017/043694

§ 371 Date: Jan. 25, 2019

Related U.S. Application Data
(60) Provisional application No. 62/366,246, filed on Jul. 25, 2016.

(51) Int. Cl. A61K 51/08; C07K 14/475; A61K 9/00; C07B 59/00

(52) U.S. Cl. CPC A61K 51/088; C07B 59/008; A61K 9/0019; C07K 14/4753

(57) ABSTRACT

Described herein are novel methods for the synthesis of radiolabeling synthons such as [¹⁸F]fluoronicotinic acid-2,3,5,6-tetrafluorophenyl ester, and also methods of labeling a protein or peptide comprising a free amine group. A novel c-Met binding peptide, and imaging methods, are also described.

### BACKGROUND

Positron emission tomography (PET) is one of the most powerful clinically established noninvasive imaging modalities, which provides not only information on biochemical, physiological and pharmacological processes, but also offers the opportunity to study the pharmacokinetics, metabolism, and mechanisms of action of novel and established drugs. Among the available PET radionuclides, fluorine-18 is favored for in vivo imaging as it offers the most suitable nuclear and chemical properties and exhibits minimal perturbation to drug structure when substituted on to low molecular weight drugs.

With the development of specific targeted peptides and proteins thru phage display library sorting to biomarkers of human disease, there is a clear need for a reliable and facile fluorine-18 radiosynthetic method to label these peptides or proteins for clinical applications. Although there are few reports of direct fluorine-18 labeling of peptides, fluorine-18 labeling of peptides and proteins is mostly done by an indirect approach using different fluorine-18 labeled small molecules. Therefore, it is important to have a convenient synthetic method to prepare a labeled synthon in high yield in a short time. Fluorine-18 radiolabeled fluoronicotinic acid-2,3,5,6-tetrafluorophenyl ester is one of the most useful synthons to radiolabel protein and peptides and has been used to label a peptide targeting c-MET.

The receptor tyrosine kinase c-MET is over expressed or mutated in various human cancers. Under normal conditions, HGF (the natural ligand) interacts with HGF or MET receptors regulating cell proliferation, motility, survival, and morphogenesis. HGF and MET signaling is essential for early development (embryogenesis) and homeostasis in adulthood and implicated in promoting tissue repair and regeneration. When “dysregulation” of this signaling pathway occurs, increased proliferation and angiogenesis, inhibition of apoptosis, and progression of metastatic disease have been observed in many human cancers. For these reasons development of tyrosine kinase inhibitors that would prevent activation of the c-Met pathway have emerged as potential therapeutics. The development of imaging probes for c-Met would also aid in evaluating responses to these targeted therapies. A PET imaging probe capable of detecting these receptors would be useful not only for diagnosis and determining the appropriate course of therapy but also for monitoring the patient response to therapy.

What is needed are new imaging probes for c-MET, methods of imaging MET expressing tumors, as well as new methods of preparing precursors and labeling probes with fluorine-18 for use in PET imaging.

### BRIEF SUMMARY

In an aspect, an 18-fluorine labeled c-Met peptide comprises Compound 1

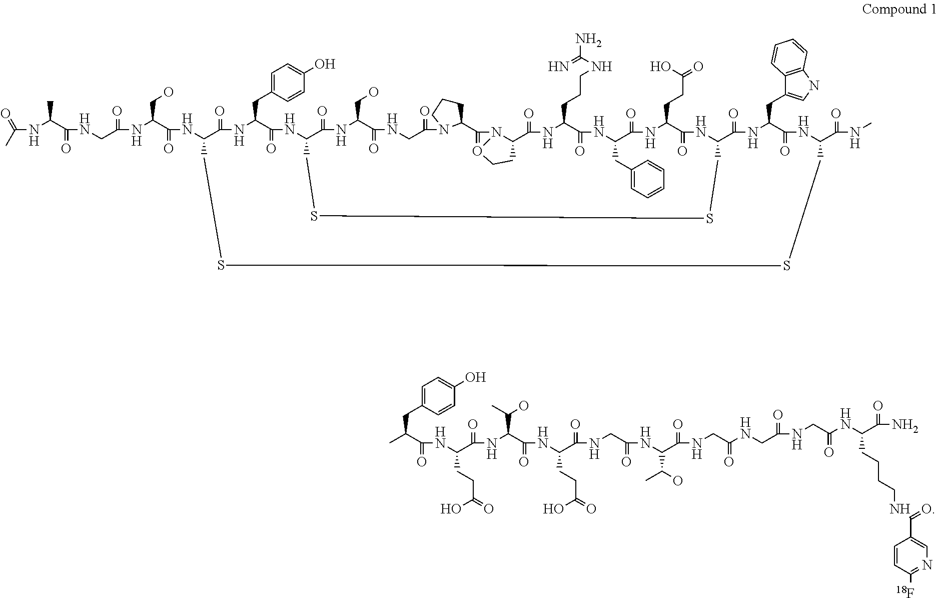

In another aspect, a composition comprises Compound 1

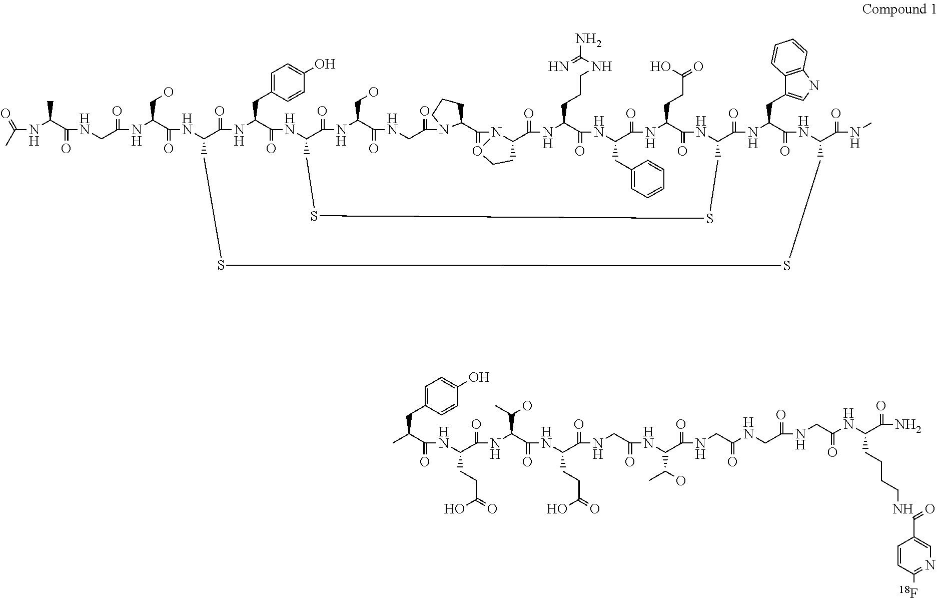

and a carrier.

In another aspect, an imaging method comprises

administering to a subject in need of c-MET imaging a detectable quantity of Compound 1

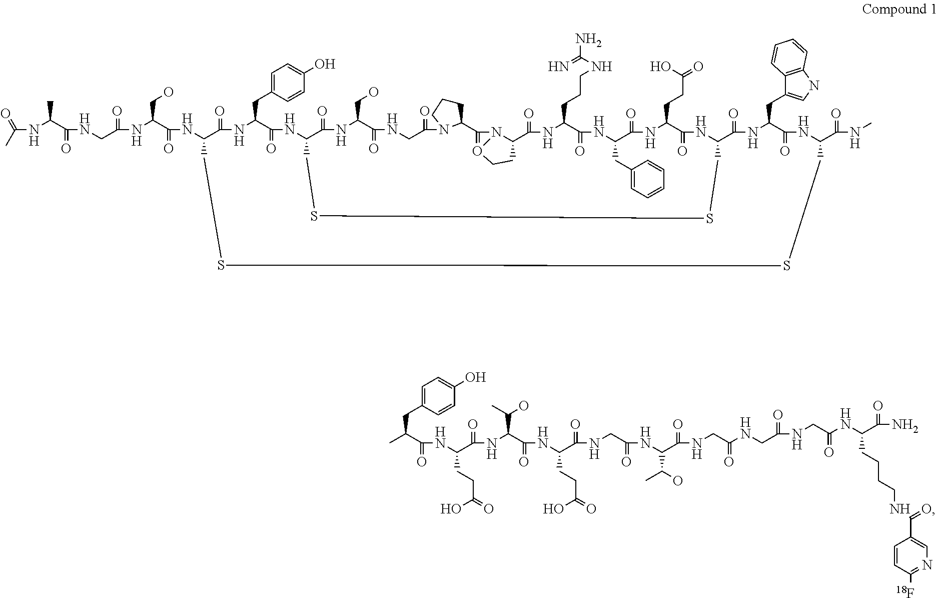

and

imaging at least a portion of the subject.

In another embodiment, a base-free method of preparing a fluorine-18-labeled ester of Compound 5, comprises

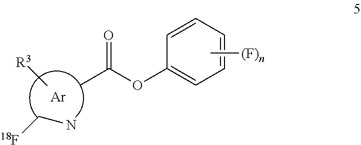

binding [¹⁸F]fluoride to an anion exchange column,

eluting the [¹⁸F] by passing a solution containing Compound 4

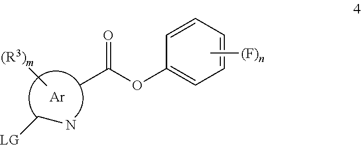

and a solvent through the anion exchange column comprising the [¹⁸F], to provide Compound 5, wherein no base is present during eluting, and wherein

LG is a leaving group, and is —NO₂, —Br, —Cl, —I, or a group of the formula —Y⁺X⁻ wherein Y is —NR¹₃ or —IR² wherein R¹ is a C₁₋₆ hydrocarbyl, preferably a C₁₋₄ alkyl, and R² is aryl, and X is Br, I, BF₄, O₂CCF₃, ClO₄, OSO₂CF₃, OSO₂C₆H₄CH₃, or —OSO₂CH₃,

R³ is NO₂, CN, or F,

the group

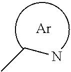

is a C₄₋₇ cyclic aromatic group wherein the bond to the tetra-substituted amine is located on a carbon adjacent to the ring nitrogen,

m is 0 to 3, provided that the valence of the group

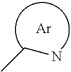

is not exceeded, and

n is 2 to 5.

In another embodiment, a method of 18-fluorine labeling a protein, peptide, or small molecule comprising a free amine group comprises

binding [¹⁸F]fluoride to an anion exchange column,

eluting the [¹⁸F] by passing a solution containing Compound 4

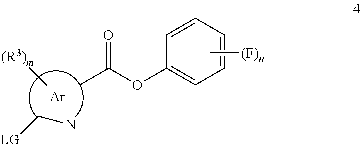

and a first solvent through the anion exchange column comprising the [¹⁸F] to provide Compound 5

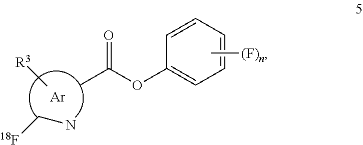

wherein no base is present during eluting; and

reacting the [¹⁸F]fluoronicotinic acid-2,3,5,6-tetrafluorophenyl ester and the protein, peptide, or small molecule comprising a free amine group in the presence of a second solvent and a base to provide the 18-fluorine labeled protein or peptide, wherein

LG is a leaving group, and is —NO₂, —Br, —Cl, —I, or a group of the formula —Y⁺X⁻ wherein Y is —NR¹₃ or —IR² wherein R¹ is a C₁₋₆ hydrocarbyl, preferably a C₁₋₄ alkyl, and R² is aryl, and X is Br, I, BF₄, O₂CCF₃, ClO₄, OSO₂CF₃, OSO₂C₆H₄CH₃, or OSO₂CH₃,

R³ is NO₂, CN, or F,

the group

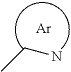

is a C₄₋₇ cyclic aromatic group wherein the bond to the tetra-substituted amine is located on a carbon adjacent to the ring nitrogen,

m is 0 to 3, provided that the valence of the group

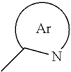

is not exceeded, and

n is 2 to 5.

### BRIEF DESCRIPTION OF THE DRAWINGS

FIG. 1 compares Compound 1 to prior art compound [¹⁸F]AH113804.

FIG. 2 shows an HPLC analysis of the crude reaction mixture of Compound 3 prepared by the inventive Sep-Pak® method. Solid line, in-line radiodetector; dotted line, UV detector at 254 nm.

FIG. 3 shows an HPLC analysis of Compound 3 prepared following the literature method. Solid line, in-line radiodetector; dotted line, UV detector at 254 nm.

FIG. 4 shows the structure of fluorine-18 labeled cyclic RGD and [¹⁸F]DCFPyL.

FIG. 5 shows an HPLC analysis of Sep-Pak® purified [¹⁸F]c(RGDfK).

FIG. 6 shows HPLC purified [18F]c(RGDfK).

FIG. 7 shows an HPLC analysis of [¹⁸F]c(RGDfK), coinjected with the nonradioactive standard. HPLC condition for FIGS. 5, 6, and 7: Agilent Eclipse plus C18 column (4.6×150 mm, 3.5 μm), mobile phase: 10%-50% in 8 minutes, 50%-90% in 15 minutes. A=acetonitrile (0.1% TFA), B=water (0.1% TFA), with a flow rate of 1.0 mL/min. Solid line, in-line radiodetector; dotted line, UV detector at 254 nm.

FIG. 8 shows an HPLC analysis of [¹⁸F]DCFPy.

FIG. 9 shows [¹⁸F]DCFPyL coinjected with the nonradioactive standard. HPLC condition for FIGS. 8 and 9: Agilent eclipse plus C18 column (4.6×150 mm, 3.5 μm), mobile phase: 5% acetonitrile in 0.1 M ammonium formate (pH 3.5), with a flow rate of 1.0 mL/min. Solid line, in-line radiodetector; dotted line, UV detector at 254 nm.

FIG. 10 shows an HPLC analysis of [¹⁸F]RSA. HPLC condition: Agilent GF250 column (9.4×250 mm, 3.5 μm), mobile phase: PBX 1×, pH 7.4, with a flow rate of 1.0 mL/min. Solid line, in-line radiodetector; dotted line, UV detector at 254 nm.

FIG. 11 shows the biodistribution of Compound 1 in MKN-45 (high Met) xenografts after 30, 60 and 120 minutes. Each bar represents % ID/g±SD of [¹⁸F] NE Met peptide [n=5)]. Compound 1 is highly retained in MKN-45 tumors (high Met expression) and rapidly cleared from non-target tissue.

FIG. 12 shows that tumor uptake of Compound 1 was significantly blocked (66%) with unlabeled Met peptide in MKN-45 xenografts. Biodistribution of Compound 1 in MKN-45 xenografts injected with Compound 1 only or a coinjection of Compound 1+unlabeled Met peptide at 60 min. Each bar represents % ID/g±SD of Compound 1 [n=5)]. Compound 1 is highly retained in MKN-45 tumors (high Met expression) and rapidly cleared from non-target tissue.

FIG. 13 shows the biodistribution of Compound 1 in U87-MG (low Met) xenografts at 1 h and 2 h. Each bar represents % ID/g±SD of Compound 1 [n=5)]. As expected, low Met expressing U87 tumor uptakes (1.6 to 0.09% ID/g) were decreased 3 to 40 fold compared to the MKN-45 high Met expressing tumors.

FIG. 14 shows that Compound 1 distinguished MET levels in vivo in human tumor mouse xenograft models. Tumor:Muscle ratios (T:M) were determined from mouse biodistributions at 30, 60 and 120 min. Each bar represents % ID/g±SD of Compound 1 [n=5)]. MKN tumors had the highest T:M of 11:1 (30 min), 56:1 (60 min) and 100:1 (120 min) while moderate Met expressing SNU-16 tumors T:M (7:1 to 18:1) and U87 T:M (3:1 to 5:1) were decreased from 2 to 60 fold over the same time course. MKN T:M ratios obtained from xenografts blocked with unlabeled Met peptide were decreased by 65% compared to unblocked.

FIG. 15 shows coronal PET images of MKN-45 and SNU-16 xenograft mice injected with Compound 1. Representative images of MKN-45 and SNU-16 xenografts at 30, 60, and 120 min post injection of Compound 1. Tumors (on shoulder) were discerned as early as 30 min.

FIG. 16 shows coronal PET images of MKN-45 xenograft mouse at 30′, 60′ and 120′ post injection of [¹⁸F]AH113804. Representative images of MKN-45 and SNU-16 xenografts at 30, 60, and 120 min post injection of [¹⁸F]AH113804. Although tumors could be discerned as early as 30 min, MKN and SNU tumor uptakes were lower with higher non-target uptakes (kidneys, lungs, and liver) compared to Compound 1 (FIG. 15) at similar times.

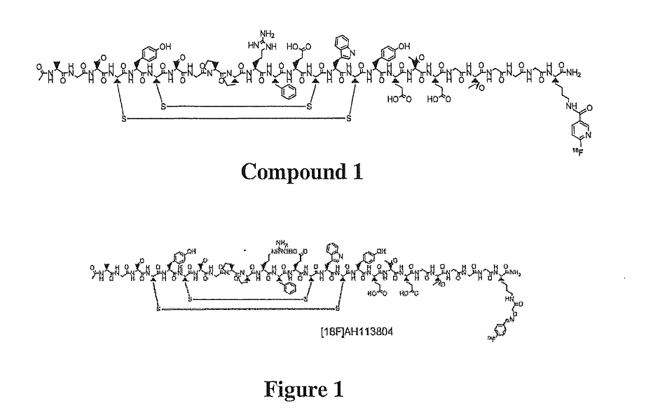

The above-described and other features will be appreciated and understood by those skilled in the art from the following detailed description, drawings, and appended claims.

### DETAILED DESCRIPTION

Various peptides that bind to c-MET are described in U.S. Pat. No. 9,000,124. A specific peptide called [¹⁸F]AH113804 was developed, and asserted to be useful for PET imaging of c-MET. The inventors of the present application, however, have found that [¹⁸F]AH113804 is challenging to isolate in pure form, and also have been unable to show that [¹⁸F]AH113804 specifically binds c-MET. The inventors of the present application have thus developed new ¹⁸F-labeled c-Met peptides and methods of 18-fluorine labeling peptides that provide both improved reagent purity and specific c-MET binding. The methods can also be used to label other peptides with short reaction times and high radiochemical yields.

In an aspect, an ¹⁸F-labeled c-Met peptide comprises Compound 1.

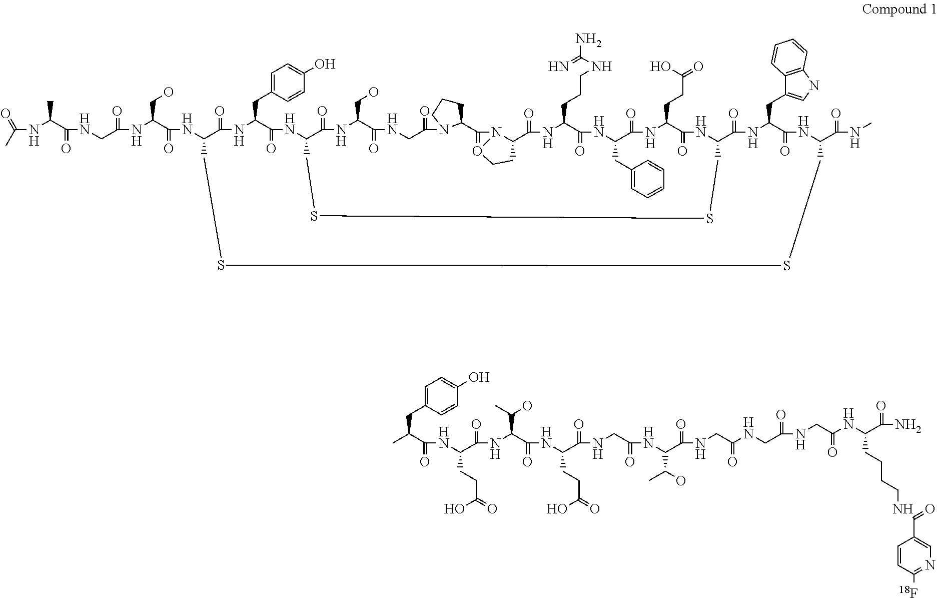

As used herein, a “c-Met peptide” is a peptide that specifically binds MET receptors in vitro and preferably in vivo.

A composition comprises Compound 1 and a carrier, which can be aqueous or non-aqueous.

Examples of non-aqueous carriers are propylene glycol, polyethylene glycol, vegetable oil, and injectable organic esters such as ethyl oleate. Aqueous carriers include water, alcoholic/aqueous solutions, saline solutions, phosphate buffered saline, parenteral vehicles such as sodium chloride, Ringer's dextrose, etc. Intravenous vehicles can include fluid and nutrient replenishers. Preservatives include antimicrobials, antioxidants, chelating agents, and inert gases. The pH and exact concentration of the various components of the pharmaceutical composition are adjusted according to routine skills in the art. In an embodiment, the composition is a composition for injection.

In an embodiment, an imaging method comprises administering to a subject in need of c-MET imaging a detectable quantity of Compound 1, and imaging at least a portion of the subject. Subjects in need of c-MET imaging include subjects in need of evaluation of c-MET expression such as subjects with a tumor that expresses c-MET or a tumor that contains c-MET mutations and/or subjects who have been treated with a c-MET targeted therapeutic. For example, it has been shown that c-MET is associated with breast cancer progression, particularly basal-like breast cancer and triple negative breast cancer, and c-MET overexpression has also been identified in non-small cell lung carcinoma, glioblastoma, gastric cancer, ovarian cancer, pancreatic cancer, thyroid cancer, head and neck cancers, colon cancer and kidney cancer. Cancer therapies that target c-MET include MET kinase inhibitors and HGF inhibitors.

A “subject” is a mammal, specifically a human, and most specifically a human having or suspected of having a tumor that expresses c-MET.

A “detectable quantity” means that the amount of the compound (e.g., Compound 1) that is administered is sufficient to enable detection of binding of the compound to c-MET. An “imaging effective quantity” means that the amount of the compound that is administered is sufficient to enable imaging of the compound bound to c-MET.

Generally, the dosage of Compound 1 can vary depending on considerations such as age, condition, sex, and extent of disease in the patient, contraindications, if any, concomitant therapies and other variables, to be adjusted by a physician skilled in the art. Dosage can vary from 0.001 μg/kg to 10 μg/kg, specifically 0.04 μg/kg to 1.4 μg/kg.

Administration to the subject can be local or systemic and accomplished intravenously, intra-arterially, intrathecally (via the spinal fluid) or the like. Administration can also be intradermal or intracavitary, depending upon the body site under examination. After administration of Compound 1, the area of the subject under investigation is examined by imaging techniques such as PET imaging techniques. The exact protocol can vary depending upon factors specific to the subject, as noted above, and depending upon the body site under examination, method of administration and type of label used; the determination of specific procedures would be routine to the skilled artisan. Blood sampling can accompany imaging to allow for measurement of the arterial input function of the radioligand. These PET and blood measurements can then be used by well-known biomathematical techniques to quantify c-MET density in areas of interest.

More specifically, Compound 1 can be used in non-invasive nuclear medicine imaging techniques such as PET imaging. Imaging is used to quantify c-MET in vivo. The term “in vivo imaging” refers to a method that permits the detection of a labeled c-MET binding compound as described herein. For nuclear medicine imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound.

Also included herein are methods of preparing precursors for the preparation of ¹⁸F-labeled proteins, peptides and small molecules, and also methods for the preparation of ¹⁸F-labeled proteins, peptides and small molecules such as Compound 1. Fluorine-18 substitution can be performed by electrophilic fluorination with ¹⁸F₂ or by nucleophilic fluorination with [¹⁸F]fluoride. In electrophilic fluorination, ¹⁸F₂ is produced along with non-radioactive fluorine gas as a carrier, so radiopharmaceuticals prepared using ¹⁸F₂ have low specific activities, because only half of the activity of ¹⁸F₂ can be electrophilically substituted. The most useful route to obtain ¹⁸F-labeled compounds of high specific activity has been via nucleophilic fluorination by a no-carrier-added [¹⁸F]fluoride. The first step of the nucleophilic fluorination process is to pass fluorine-18 containing target water through an anion exchange resin to trap the activity as [¹⁸F]fluoride. The activity can be eluted as [¹⁸F]-salt from the anion exchange resin with a base solution. The base solution can be any suitable inorganic or organic base, for example an alkali metal or alkaline earth metal base, or a tetraalkyl ammonium or phosphonium hydroxide. In some embodiments, the eluted [¹⁸F]fluoride salt can be [¹⁸F]KF, [¹⁸F]CsF, or [¹⁸F]tetrabutyl ammonium fluoride (TBAF). The next step is to dry the activity with acetonitrile (1 mL×3, azeotropic drying). This azeotropic drying takes 15-20 minutes with some loss of activity due to normal decay and evaporation. The dried [¹⁸F]-salt and base mixture is then heated with the precursor to be labeled at elevated temperature (40-180° C.) in an organic solvent medium to obtained fluorine-18 labeled tracers. Many precursors cannot withstand the temperatures in the highly basic medium. This multistep and harsh fluorine-18 labeling procedure restricts the access to the many useful fluorine-18 labeled PET imaging agents. Various modifications have been made to improve these standard protocols, such as the use of ionic liquid media or various additives, but these modifications have not been widely accepted. Therefore, there is a clear need for the development of faster and milder nucleophilic fluorination method for the extended use of fluorine-18 PET tracers in nuclear medicine.

Fluorine-18 radiolabeled fluoronicotinic acid-2,3,5,6-tetrafluorophenyl ester is a very useful synthon to radiolabel temperature sensitive biomolecules. This was first reported by Olberg et al (Olberg D E, Arukwe J M, Grace D, Hjelstuen O K, Solbakken M, Kindberg G M, et al. “One Step Radiosynthesis of 6-[F-18]Fluoronicotinic Acid 2,3,5,6-Tetrafluorophenyl Ester ([F-18]F-Py-TFP): A New Prosthetic Group for Efficient Labeling of Biomolecules with Fluorine-18.” Journal of Medicinal Chemistry 2010; 53:1732-40.) Since then, [F-18]F-Py-TFP has been used by the present inventors and other groups to radiolabel proteins and peptides. However, the precursor is not stable in K₂₂₂/K₂CO₃. This issue was overcome by using less basic tetrabutyl ammonium bicarbonate (TBA-HCO₃), but due to the limited amount of base used in, there was a significant amount of loss of radioactivity.

While searching for a better procedure, the inventors have discovered an unprecedented fluorine-18 labeling technique to provide this prosthetic group. This method eliminates loss of activity due to evaporation and normal decay. Unexpectedly, the [¹⁸F]fluoride activity from the anion-exchange column (e.g., Sep-Pak®) can be eluted by a quaternary ammonium triflate precursor (Compound 2, 4), to provide the eluted fluorine-18 labeled product (compound 3, 5). (See Scheme 1) Nucleophilic fluoride substitution occurred inside the anion-exchange column instantly at room temperature.

A specific embodiment of the method is shown in Scheme 1:

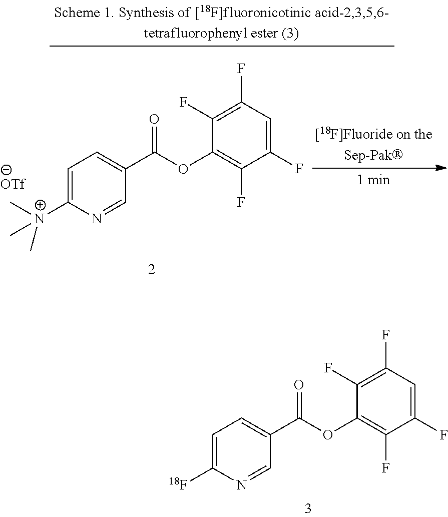 [SMILES: C[N+](C)(C)c1ccc(C(=O)Oc2c(F)c(F)cc(F)c2F)cn1 ; O=C(Oc1c(F)c(F)cc(F)c1F)c1ccc([18F])nc1]

Scheme 2 provides a broader embodiment of the method:

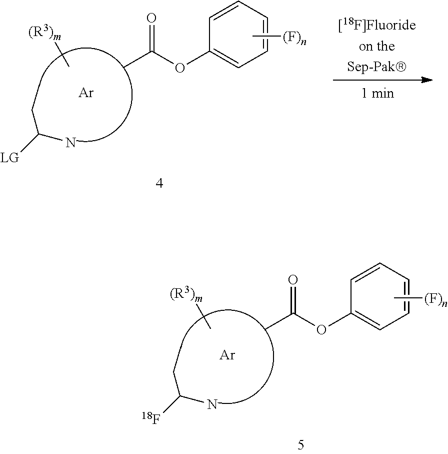

wherein

LG is a leaving group, and is —NO₂, —Br, —Cl, —I, or a group of the formula —Y⁺X⁻ wherein Y is —NR¹₃ or —IR² wherein R¹ is a C₁₋₆ hydrocarbyl, preferably a C₁₋₄ alkyl, and R² is aryl, and X is Br, I, BF₄, O₂CCF₃, ClO₄, OSO₂CF₃, OSO₂C₆H₄CH₃, or OSO₂CH₃,

R³ is NO₂, CN, or F,

the group

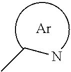

is a C₄₋₇ cyclic aromatic group wherein the bond to the tetra-substituted amine is located on a carbon adjacent to the ring nitrogen,

m is 0 to 3, provided that the valence of the group

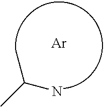

is not exceeded, and

n is 2 to 5.

Additional specific compounds of Formula 4 include

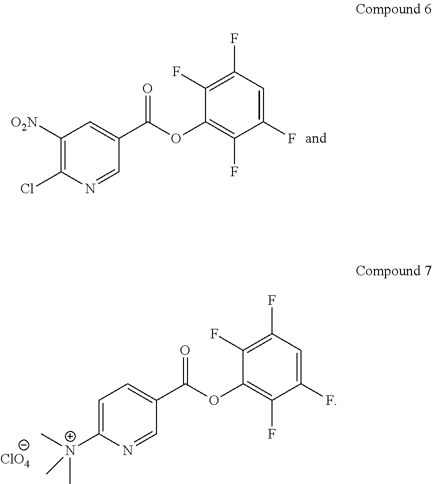 [SMILES: O=C(Oc1c(F)c(F)cc(F)c1F)c1cnc(Cl)c([N+](=O)[O-])c1 ; "ClO4" ; C[N+](C)(C)c1ccc(C(=O)Oc2c(F)c(F)cc(F)c2F)cn1]

The labeled biomolecule (e.g., protein, peptide or small molecule) is illustrated below:

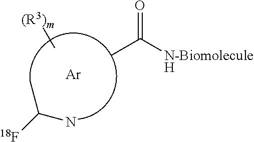

wherein

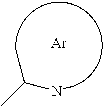

and m are as described in Compound 5.

In an embodiment, a base-free method of preparing an [¹⁸F]fluoroaromatic acid-2,3,5,6-tetrafluorophenyl ester (Compound 5) comprises, consists essentially of, or consists of binding [¹⁸F]fluoride to an anion exchange column; eluting the [¹⁸F] by passing a solution containing Compound 4 and a solvent through the anion exchange column comprising the [¹⁸F], wherein eluting provides the [¹⁸F]fluoroaromatic acid-2,3,5,6-tetrafluorophenyl ester, and wherein no base is present during eluting.

In one embodiment, Compound 4 is N,N,N-trimethyl-5-((2,3,5,6-tetrafluorophenoxy)carbonyl)pyridin-2-aminium trifluoromethanesulfonate and Compound 5 is [¹⁸F]fluoronicotinic acid-2,3,5,6-tetrafluorophenyl ester.

In an embodiment, a base-free method of preparing [¹⁸F]fluoronicotinic acid-2,3,5,6-tetrafluorophenyl ester (Compound 3) comprises, consists essentially of, or consists of binding [¹⁸F]fluoride to an anion exchange column; eluting the [¹⁸F] by passing a solution containing N,N,N-trimethyl-5-((2,3,5,6-tetrafluorophenoxy)carbonyl)pyridin-2-aminium trifluoromethanesulfonate (Compound 2) and a solvent through the anion exchange column comprising the [¹⁸F], wherein eluting provides the [¹⁸F]fluoronicotinic acid-2,3,5,6-tetrafluorophenyl ester, and wherein no base is present during eluting.

In an embodiment, eluting is performed in five minutes or less, two minutes, or less, or, preferably, one minute or less. Any suitable solvent for compounds 2-5 can be used. Polar solvents are generally preferred, which can be protic or aprotic. In some embodiments, the solvent comprises acetonitrile, t-butanol, dimethyl sulfoxide, or a combination thereof. In a preferred embodiment, the solvent does not contain water. Although the binding or elution can be performed at any suitable temperature, for example up to 40° C., in a preferred embodiment, the fluorination reaction and eluting are performed at room temperature.

In a conventional method, during aromatic fluorination, the first step is to pass the ¹⁸F over an anion exchange column, then elute and dry the ¹⁸F in the presence of base, which generally takes 15-20 minutes. In the present method, the ¹⁸F labeling is achieved without elution and azeotropic drying of [¹⁸F]fluoride in the presence of base. Fluorinating while the ¹⁸F is retained on the anion exchange column saves time and reduces loss of activity due to drying and normal decay.

The [¹⁸F]fluoronicotinic acid-2,3,5,6-tetrafluorophenyl ester can be used to label proteins, peptides and small molecules containing a free amine. In an embodiment, the peptide is a c-Met peptide, a PSMA targeting small molecule, an RGD peptides, or albumin.

In another embodiment, a method of 18-fluorine labeling a protein, peptide or small molecule comprises, consists essentially of, or consists of binding [¹⁸F]fluoride to an anion exchange column, eluting the [¹⁸F] by passing a solution containing Compound 4 and a first solvent through the anion exchange column comprising the [¹⁸F], wherein eluting provides Compound 5, and wherein no base is used to produce Compound 5; and reacting Compound 5 and a protein, peptide or small molecule comprising a free amine group in the presence of a second solvent and a base to provide the 18-fluorine labeled protein or peptide.

In yet another embodiment, a method of 18-fluorine labeling a protein, peptide or small molecule comprises, consists essentially of, or consists of binding [¹⁸F]fluoride to an anion exchange column, eluting the [¹⁸F] by passing a solution containing N,N,N-trimethyl-5-((2,3,5,6-tetrafluorophenoxy)carbonyl)pyridin-2-aminium trifluoromethanesulfonate and a first solvent through the anion exchange column comprising the [¹⁸F], wherein eluting provides the [¹⁸F]fluoronicotinic acid-2,3,5,6-tetrafluorophenyl ester, and wherein no base is used to produce the [¹⁸F]fluoronicotinic acid-2,3,5,6-tetrafluorophenyl ester; and reacting the [¹⁸F]fluoronicotinic acid-2,3,5,6-tetrafluorophenyl ester and a protein, peptide or small molecule comprising a free amine group in the presence of a second solvent and a base to provide the 18-fluorine labeled protein, peptide or small molecule.

In an embodiment, eluting is performed in five minutes or less, two minutes, or less, or, preferably, one minute or less. Any suitable solvent for compounds 2 and 4 can be used as the first solvent. Polar solvents are generally preferred, which can be protic or aprotic. In some embodiments, the solvent comprises acetonitrile, t-butanol, dimethyl sulfoxide, or a combination thereof. In a preferred embodiment, the first solvent does not contain water. Although the binding or elution can be performed at any suitable temperature, for example up to 40° C., in a preferred embodiment, the fluorination reaction and eluting are performed at room temperature.

Any suitable solvent for Compound 5, [¹⁸F]fluoronicotinic acid-2,3,5,6-tetrafluorophenyl ester and the protein or peptide can be used as the second solvent. Polar aprotic solvents are generally preferred. Exemplary second solvents include organic solvents such as dimethyl formamide (DMF), dimethyl sulfoxide (DMSO), acetonitrile, dimethylacetamide, N-methylpyrrolidone (NMP), and aqueous solvents such as acetonitrile-water, aqueous phosphate buffer, and the like. Exemplary bases include secondary and tertiary amines, for example N,N-diisopropylethylamine (DIPEA), triethyl amine, and inorganic bases such as NaHCO₃, and the like. The reaction temperature is typically 40 to 60° C., and the reaction time is typically 10 to 15 min.

The inventors have also used the new methods described herein to prepare [¹⁸F]c(RGDfK), [¹⁸F] DCFPyL, and [¹⁸F]albumin in short synthesis times (30-50 min) with moderate to high radiochemical yields. For the first time RGD-peptide c(RGDfK) has been radiolabeled with Compound 3. This method is comparable with direct fluorine-18 labeling approaches. Because of the simplicity of the method, it could easily be automated for routine clinical production.

The invention is further illustrated by the following non-limiting examples.

### EXAMPLES

### Example 1

### Radiosynthesis of [¹⁸F]fluoronicotinic acid-2,3,5,6-tetrafluorophenyl ester (3)

### Materials and Methods

Tetrabutylammonium hydrogen carbonate (0.075 M) used for radiochemical work was purchased from ABX (Radeberg, Germany). All other chemicals and solvents were received from Sigma Aldrich® (St. Louis, Mo., USA) and used without further purification. The precursor N,N,N-trimethyl-5-((2,3,5,6-tetrafluorophenoxy)carbonyl)pyridin-2-aminium trifluoromethanesulfonate (2) and cold standard fluoronicotinic acid-2,3,5,6-tetrafluorophenyl ester were prepared according to methods known in the art. Fluorine-18 was purchased from National Institutes of Health cyclotron facility (Bethesda, Md., USA). Chromafix® 30-PS-HCO₃ anion-exchange cartridge was purchased from Macherey-Nagel (Duren, Germany). Columns and all other the Sep-Pak® cartridges used in this synthesis were obtained from Agilent Technologies (Santa Clara, Calif., USA) and Waters (Milford, Mass., USA), respectively. Oasis® MCX Plus cartridge was conditioned by passing 5 mL ethanol, 10 mL air and 10 mL water. Analytical HPLC analyses for radiochemical work were performed on an Agilent 1200 Series instrument equipped with multi-wavelength detectors using an Agilent Eclipse XDB C18 column (4.6×150 mm, 5 μm). Mobile phase: 20-80% acetonitrile (0.1% TFA) in water (0.1% TFA) in 12 min with a flow rate of 1.0 mL/min

### Results

Precursor and cold standard were synthesized by methods known in the art. Fluorine-18 labeling was achieved on the anion exchange column (Sep-Pak®) (Scheme 1). Specifically, fluorine-18 containing target water from the cyclotron was diluted with 2 mL water (10-25 mCi) and passed through an anion exchange cartridge (Sep-Pak®; Chromafix® 30-PS-HCO₃), resulting in binding of the [¹⁸F]fluoride to the column. The column was washed with 3 mL anhydrous acetonitrile. Over 70% activity was incorporated in to the product (3) by passing 10 mg of quaternary ammonium triflate precursor (2) in 1 ml acetonitrile through the Sep-Pak® in 1 min. Fluoride incorporation efficiency was tested using different conditions (Table 1). Better elution of the product was observed with a mixture of solvents (2:8 acetonitrile, t-butanol). A slight improvement of yield was observed with an increase in precursor amount (15 mg). No significant improvement of yield was observed with further dilution of the precursor (2 mL). The entire process was performed at room temperature.

TABLE 1
Elution conditions from the Sep-Pak ® to prepare [¹⁸F] 3

| Amount of precursor 3 | Solvent | Eluted from the Sep- |
|---|---|---|
| (mg) | (1 mL) | Pak ® (%)^{a} |
| 15 | Acetonitrile | 75 ± 3^{b} |
| | 2:8, acetonitrile:t-butanol | 83 ± 2^{b} |
| | DMSO | 47 |
| 10^{c} | 2:8, acetonitrile:t-butanol | 67 |
| 10 | Acetonitrile | 72 ± 1^{b} |
| | 2:8, acetonitrile:t-butanol | 78 ± 3^{b} |
| | DMSO | 34 |
| 5 | Acetonitrile | 59 |
| | 2:8, acetonitrile:t-butanol | 57 |
| | DMSO | 24 |
| 3 | Acetonitrile | 30 ± 2^{b} |

^{a}Radiolabeling was carried out with 10-20 mCi of fluorine-18;
^{b}n = 3;
^{c}Literature method

In this new method, fluorine-18 labeling was achieved without azeotropic drying of [¹⁸F]fluoride. This process saved 15-20 min in comparison to the conventional nucleophilic radiolabeling method. Therefore, the loss of activity due to evaporation and normal decay is negligible. Moreover, as no base is used and fluorination proceeds at room temperature, the stability of the precursor in basic medium and/or at high temperature will not be an issue.

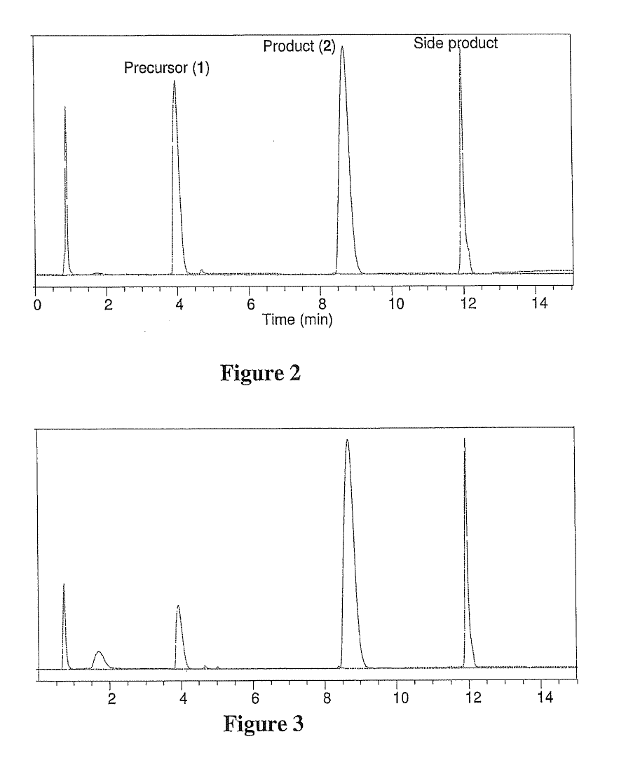

An HPLC chromatogram of the crude product (FIG. 2) prepared using the Sep-Pak® reaction technique was almost identical with that of compound 3 prepared following the literature method (FIG. 3). The peak at approximately 4 minutes is for the precursor and approximately 12 minutes is the side product bis(2,3,5,6-tetrafluorophenyl) pyridine-2,5-dicarboxylate. The quantification of the side product was not performed but from relative HPLC integration ratio of the precursor to side product it is obvious that side product is less for the current method compared to the literature method (1:0.6 vs. 1:2).

The overall radiochemical yield was 72±3% (uncorrected, n=3) in a 5 min synthesis time with a radiochemical purity of >98% by analytical HPLC. The identity of the product (3) was further confirmed by comparing its HPLC retention time with co-injected, authentic nonradioactive fluoronicotinic acid-2,3,5,6-tetrafluorophenyl ester (data not shown).

### Example 2

### Fast Indirect fluorine-18 Labeling of Protein/Peptide Using 6-fluoronicotinic Acid-2,3,5,6-tetrafluorophenyl Prosthetic Group

### Materials and Methods

PSMA precursor, di-tert-butyl (((S)-6-amino-1-(tert-butoxy)-1-oxohexan-2-yl)carbamoyl)-L-glutamate formate salt, and cold standard were prepared according to methods known in the art. Cyclic peptide c(RGDfK) was obtained from Peptides International Inc. (Louisville, Ky., USA). PBS 1× buffer (Gibco) was obtained from Life Technologies (Carlsbad, Calif., USA). Normal saline was obtained from Quality Biological (Gaithersburg, Md., USA). PD10 MiniTrap™ columns were obtained from GE Healthcare Bioscience (Pittsburg, Pa., USA). All other chemicals and solvents were received from Sigma-Aldrich (St. Louis, Mo., USA) and used without further purification. The precursor N,N,N-trimethyl-5-((2,3,5,6-tetrafluorophenoxy)carbonyl)pyridine-2-aminium fluoromethanesulfonate (2) and cold standard 6-fluoronicotinic acid-2,3,5,6-tetrafluorophenyl ester were prepared by known methods. Fluorine-18 was obtained from National Institutes of Health cyclotron facility (Bethesda, Md., USA). Chromafix 30-PS-HCO₃ anion exchange cartridge was purchased from Macherey-Nagel (Duren, Germany). Columns and all other Sep-Pak® cartridges used in this synthesis were obtained from Agilent Technologies (Santa Clara, Calif., USA) and Waters (Milford, Mass., USA), respectively. tC18 environmental cartridge was activated by passing 5 mL ethanol followed by 10 mL water. Oasis MCX Plus cartridge was conditioned with 5 mL anhydrous acetonitrile. Semiprep HPLC purification and analytical HPLC analyses for radiochemical work were performed on an Agilent 1200 Series instrument equipped with multiwavelength detectors. Mass spectrometry (MS) was performed on a 6130 Quadrupole LC/MS Agilent Technologies instrument equipped with a diode array detector.

Preparation of ¹⁹F standard of c(RGDfK): To a solution of c(RGDfK) (10 mg, 0.016 mmol) in acetonitrile (1 mL) and water (1 mL) was added 6-fluoronicotinic acid-2,3,5,6-tetrafluorophenyl ester (4.67 mg, 0.016 mmol) and N,N-diisopropylethylamine (6.2 mg, 0.048 mmol). The reaction mixture was stirred at 50° C. for 1 hour. The product was purified by semipreparative HPLC (conditions: Agilent Eclipse plus C18 column [9.4×250 mm, 5 μm], mobile phase: 5%-50% acetonitrile in water [0.1% trifluoroacetic acid (TFA)], with a flow rate of 4.0 mL/min.) The product peak was collected and freeze-dried to obtain the ¹⁹F cold standard of c(RGDfK) (3 mg, 27% yield). The LC/MS calculated for C₃₃H₄₃FN₁₀O₈, 726.32 found 727.20 (M+H)+.

Radiosynthesis of 6-[¹⁸F]fluoronicotinic acid-2,3,5,6-tetrafluorophenyl ester (3): Fluorine-18 labeled target water (10-25 mCi) was diluted with 2 mL water and passed through an anion-exchange cartridge (Chromafix® 30-PS-HCO3). The cartridge was washed with anhydrous acetonitrile (6 mL) and dried for 1 minute under vacuum. The [¹⁸F]fluoride from the Sep-Pak® was eluted with quaternary ammonium triflate precursor (5-7 mg, 2) in 0.5 mL 1:4, acetonitrile: t-butanol through a conditioned Oasis® MCX Plus cartridge. The cartridge was flushed with 1 mL acetonitrile and collected in the same vial for small molecule and peptide labeling. The cartridges were flushed with 2 mL diethyl ether for protein labeling.

Radiosynthesis and stability test of [¹⁸F]c(RGDfK): To the solution of 3 (1.5 mL) was added a mixture of c(RGDfK) (3-5 mg) and sodium bicarbonate (10-15 mg) in 1 mL water. The solution was stirred for 10 minutes at 50° C. The product was purified by either Sep-Pak® or semiprep HPLC. For Sep-Pak® purification, the mixture was diluted with 30 mL of water and passed through tC18 environmental cartridge. The cartridge was washed with water (10 mL) followed by 10% ethanol in water (10 mL). The product was eluted with 3 mL 30% ethanol in water. For semiprep HPLC purification, the crude reaction mixture was diluted with 2 mL HPLC buffer and injected to the HPLC (conditions: Phenomenex Luna® C18 column (10×250 mm, 5 μm), mobile phase: 25% ethanol in 50 mM o-phosphoric acid, with a flow rate of 4 mL/min). The identity and purity of the product was confirmed by analytical HPLC.

To test the serum stability, 2 mCi of [¹⁸F]c(RGDfK) was added to whole human serum (2 mL) and kept at room temperature. At different time interval (0, 1, 2 and 4 h), 20 μL of the incubated sample was directly injected to the analytical HPLC without further processing.

Radiosynthesis of [¹⁸F]DCFPyL: To the solution of 3 (1.5 mL) was added an acetonitrile solution (300 μL) of di-tert-butyl (((S)-6-amino-1-(tert-butoxy)-1-oxohexan-2-yl)carbamoyl)-L-glutamate formate salt (3-5 mg)49 with triethylamine (5 μL). The solution was stirred for 10 minutes at 50° C. Solvent was evaporated under N₂ and vacuum, and TFA (400 μL) was added. The mixture was stirred for 10 minutes at 50° C. The TFA was removed under N₂ and vacuum. Ethanol in 50 mM phosphoric acid (10%, 3 mL) was added to the crude mixture, which was purified by semiprep HPLC (conditions: Agilent Eclipse plus C18 column [9.4×250 mm, 5 μm], mobile phase: 12% ethanol in 50 mM phosphoric acid, with a flow rate of 4 mL/min). The identity and purity of the product were confirmed by analytical HPLC.

Radiosynthesis of [¹⁸F]albumin: The solvent from 3 was removed under nitrogen at 40° C. The conjugation reaction with albumin was performed according to methods known in the art. Briefly, to the vial containing 3 was added albumin (20 mg in 450 μL of phosphate buffer of pH 9+50 μL dimethylsulfoxide) and the vial was incubated for 15 min at 40° C. The product was purified by PD10 MiniTrap size exclusion column using phosphate buffer (pH 7.4) as an eluent. The product fraction was collected in 0.8 mL. Formation of the product was confirmed by analytical HPLC.

### Results and Discussion

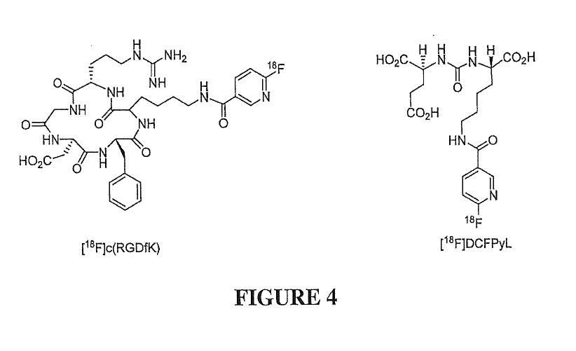

Fluorine-18 labeled 6-fluoronicotinic acid-2,3,5,6-tetrafluorophenyl ester (3) is a useful prosthetic group for radiolabeling of biomolecules. Compound 3 for this study was prepared according to the methods described herein and purified by passing through an activated Oasis MCX Plus cartridge. In this method, 3 is formed directly by passing the precursor solution (2) through the anion-exchange cartridge (Chromafix® 30-PS-HCO3). The use of anhydrous acetonitrile, dimethyl sulfoxide, or mixture of acetonitrile/t-butanol provides nucleophilic displacement to form the product. Aqueous acetonitrile, methanol, or ethanol solution of 2 only elutes the fluorine-18 from the anion exchange cartridge as a fluoride salt. Compound 3 was purified by passing through the activated Oasis MCX Plus cartridge. The activated ester was eluted from the cartridge by flushing with either acetonitrile for peptide, and small molecule labeling (FIG. 4) or diethyl ether for protein labeling.

[18F]c(RGDfK): Integrin αvβ3 is a potential molecular marker for angiogenesis during imaging and therapy due to its significant up-regulation on activated endothelial cells. The tripeptide Arg-Gly-Asp (RGD) has been extensively used as imaging tracer for integrin αvβ3 because of its high affinity and specificity. Recently, the radiosynthesis, dosimetry, pharmacokinetics, and clinical efficacy of clinically available RGD-based PET tracers. [¹⁸F]Galacto-RGD, radiolabeled by an indirect approach using 4-nitrophenyl-2-[¹⁸F]fluoropropionate, was the first fluorine-18 labeled PET tracer of this class tested clinically. There are other conventional C-¹⁸F bond containing RGD-based tracers. These tracers are prepared in multistep syntheses with several HPLC purifications, thus requiring a long synthesis time.

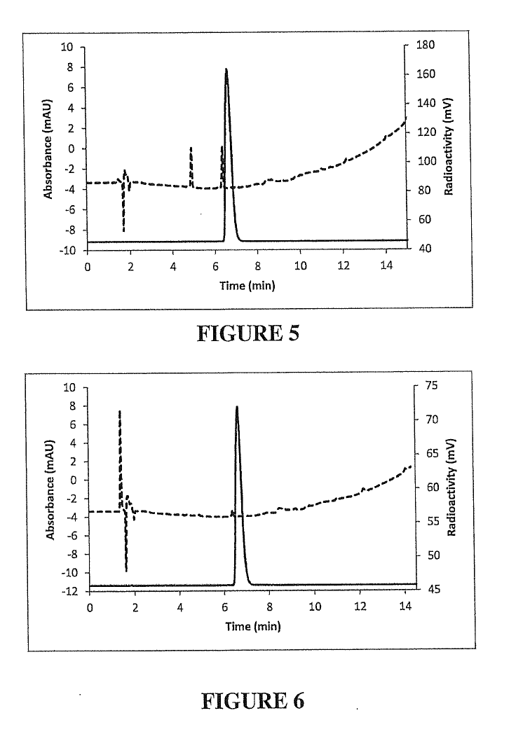

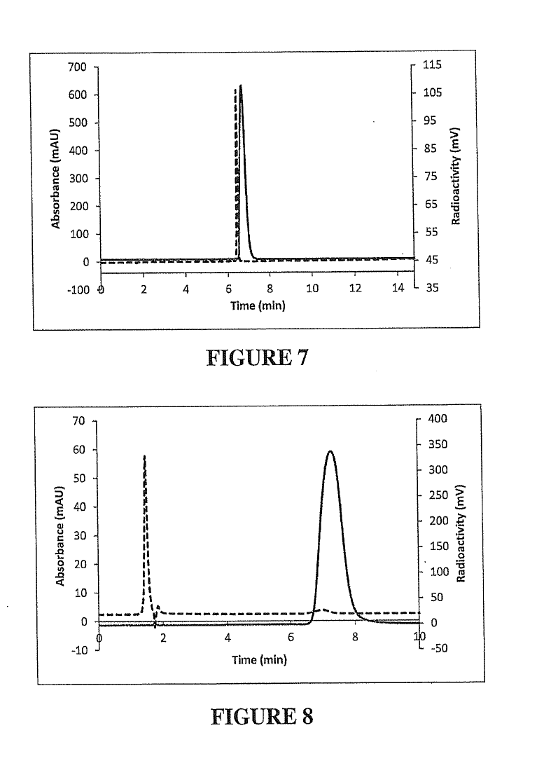

Reaction of 3 with c(RGDfK) in the presence of base (sodium carbonate) proceeded with over 80% radiochemical conversion to the product (data not shown) by analytical HPLC. The compound was purified by Sep-Pak® to produce >98% radio chemically pure (FIG. 5) product with a SA of 1000 to 2200 Ci/mmol (end of synthesis, n=12). The overall radiochemical yield was 32% to 43% (uncorrected, n=6) in a 30-minute synthesis time. A minor UV impurity peak at 5 minutes was observed in analytical HPLC (FIG. 5). In a typical radiosynthesis starting from 103 mCi of [18F]F—, the amount of impurity was <7 ug/mL in 39 mCi (3 mL) of product. The crude product was also purified by semiprep HPLC to remove the impurity peak (FIG. 6). The identity of the product [18F]c(RGDfK) was confirmed by comparing its HPLC retention time with coinjected, authentic nonradioactive standard (FIG. 7). [18F]c(RGDfK) showed excellent serum stability up to 4-hour post synthesis (data not shown).

[¹⁸F]DCFPyL: Prostate cancer (PC) is the most common cancer in men in the United States. It is the second leading cause of death from cancer in men. Therefore, over the decades, there has been an increasing interest in synthesis and evaluation of PET tracers for PC. [¹⁸F]FDG, the most widely used metabolic radiotracer for PET imaging of tumors, gave mixed results in PC. Although carbon-11 or fluorine-18 choline PET/CT showed promising results for the detection of bone metastases, this approach has limitations in terms of sensitivity and specificity. Therefore routine clinical use of carbon-11 or fluorine-18 choline PET/CT is debatable. This unmet clinical need led to the development of another class of PSMA target specific tracers. Overexpression of PSMA has been linked to PC and is an important target in patients with negative bone scan who are at high risk of metastatic disease. A recent review summarized the current use of PET tracers such as [¹¹C]choline, [¹⁸F]fluorocholine, gallium-68, and fluorine-18 labeled low-molecular weight PSMA inhibitors including DCFBC and DCFPyL in PC management. The second generation PSMA inhibitor showed high tumor: background ratio and favorable pharmacokinetics compared to other small molecules. Therefore, development of reproducible radiochemical synthesis with high radiochemical yield for this tracer is of interest. Synthesis of [¹⁸F]DCFPyL was first reported by an indirect method using 3 synthesized by the methods described herein.

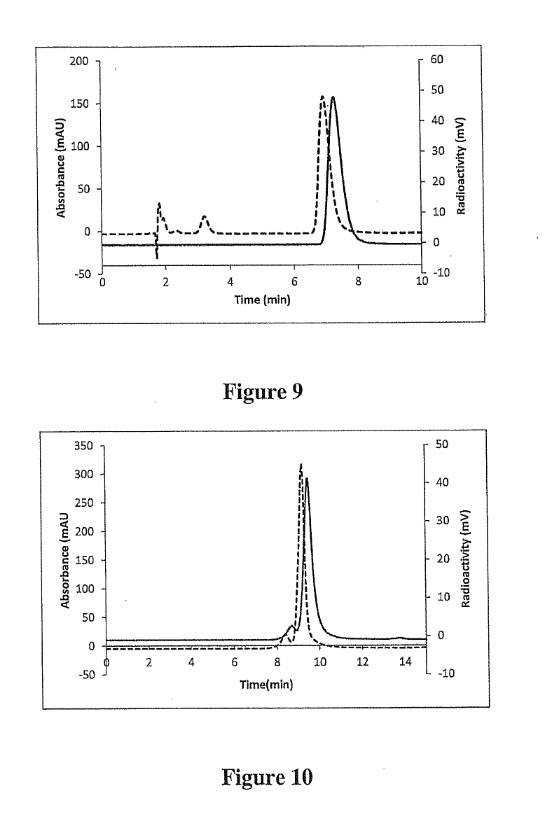

Compound 3 was prepared on a Sep-Pak® and purified by passing through an Oasis MCX plus cartridge. The cartridge efficiently removed unreacted precursor (2) from the product 3. Hence this method of preparation of 3 is comparable to initial anion exchange catch and release of fluorine-18 containing target water (Table2). Final conjugation, deprotection, and purification were done according to the literature method. The overall radiochemical yield was 25% to 32% (uncorrected, n=6) in a 45-minute synthesis time. Both radiochemical and chemical purity were >98% determined by analytical HPLC (FIG. 8) with a SA of 1200 to 2600 Ci/mmol (end of synthesis, n=15). The identity of the product was confirmed by comparing its HPLC retention time with coinjected, authentic nonradioactive standard (FIG. 9). The total labeling method is comparable with the direct method of radiolabeling (Table 2).

TABLE 2
Key steps of direct labeling method and current indirect labeling method
to prepare [18F]DCFPyL

| Direct labeling | |
|---|---|
| method (Prior Art) | Indirect radiolabeling method (Invention) |
| F-18 catch on the anion | F-18 catch on the anion exchange resin |
| exchange resin | |
| Wash with water | Wash with anhydrous acetonitrile |
| Elution with base | Drying under vacuum |
| Azeotropic drying | Elution with precursor (2) through Oasis MCX |
| Reaction with precursor | Reaction with second precursor |
| Deprotection, | Deprotection, purification |
| purification | |

In vitro binding studies with [¹⁸F]DCFPyL exhibited high affinity (nM) for prostate-specific membrane antigen (PSMA) in human prostate cancer cells with known high PSMA expression. (data not shown) In vivo [¹⁸F]DCFPyL biodistributions and PET images with xenograft mouse models using this same tumor cell line were comparable with previously reported results indicating that the biological activity had been retained. (data not shown)

[¹⁸F]albumin: Recently, fluorine-18 labeling of albumin by conjugation with 3 has been reported. The labeled albumin showed excellent blood pool imaging property. We therefore set out to further simplify the radiolabeling using the current method. By conjugating 3 to target pendant amine groups, albumin can be radiolabeled in 30 minutes with moderate radiochemical yield (Table 3). The radiochemical purity (>98%) and chemical purity (>98%) of the labeled albumin were determined by size exclusion chromatography (FIG. 10).

TABLE 3
Comparison of yield, time, and SA for prior art and current method

| | Yield (%) | Yield (%) | SA (Ci/mmol) | SA (Ci/mmol) | Time (minutes) | Time (minutes) |
|---|---|---|---|---|---|---|
| Compound | Prior Art | Current | Prior Art | Current | Prior Art | Current |
| [18F]RGD^{a} | 10-35^{b} | 39-43 | 2-2700 | 1000-2200 | 90-218 | 30 |
| [18F]DCFPyL | 5-53^{b} | 25-32 | 340-120000 | 1200-2600 | 55-128 | 45 |
| [18F]albumin | 18-35^{c} | 26-35 | n/a | n/a | 90 | 30 |

^{a}Only C-18F bonded tracers are included in this table;
^{b}decay corrected;
^{c}decay uncorrected

In summary, the yield and synthesis time of this current method has been compared with the literature reported methods for two known PET tracers ([¹⁸F]DCFPyL and [¹⁸F] Albumin) in Table 3. The RGD peptide (cRGDfK) has not been radiolabeled using 3, therefore the yield and synthesis time of this tracer is compared with the known C-¹⁸F bonded RGD tracers (Table 3). The current method requires much less time with comparable or higher radiochemical yield.

### Example 3

### Preparation of Compound 1

The peptide of SEQ ID NO: 1 was incubated with fluorine-18 radiolabeled fluoronicotinic acid-2,3,5,6-tetrafluorophenyl ester prepared according to Example 1 in DMF a base DIPEA for 10 min at 50° C. to provide Compound 1. The peptide of SEQ ID NO: 1 is a cyclic peptide including disulfide bonds Cys4-Cys16, and Cys 6-Cys14.

(SEQ ID NO: 1)
Ala-Gly-Ser-Cys-Tyr-Cys-Ser-Gly-Pro-Pro-Arg-
Phe-Glu-Cys-Trp-Cys-Tyr-Glu-Thr-Glu-Gly-Thr-
Gly-Gly-Gly-Lys

### Example 4

### Imaging of Human Gastric Carcinoma and Glioblastoma with Compound 1

Compound 1 was evaluated using human gastric carcinoma (MKN-45, SNU-16) and glioblastoma (U87-MG) cells and xenografts. Biodistribution and PET imaging studies with MK, SN or U87 xenografts were done at 30, 60, and 120 min post FMetP injections (intravenous) from which blood and tissue uptakes were determined [% injected dose/g (% ID/g)].

In vitro saturation assays were performed to determine the binding affinity of Compound 1 for c-MET receptors. Increasing concentrations of Compound 1 were incubated with MKN-45, SNU-16 or U87-MG cells. Non-specific binding was determined in the presence of an unlabeled Met peptide (10⁻⁵ M). Bound peptide was separated from free peptide and the radioactive content was determined. Data was analyzed using a one site binding hyperbola. Compound 1 exhibited high affinity (nM) and specific binding (>90%) to Met with MKN-45 cells. The binding constant was determined to be 3.9 nM. In addition, the estimated Met expression levels (2.4×10⁶ receptors per cell) were consistent with known expression in MKN-45, SNU-16, and U87-MG cells. It was further determined that Compound 1 can distinguish c-MET concentrations in SNU-16 and U87-MG cell lines. (data not shown)

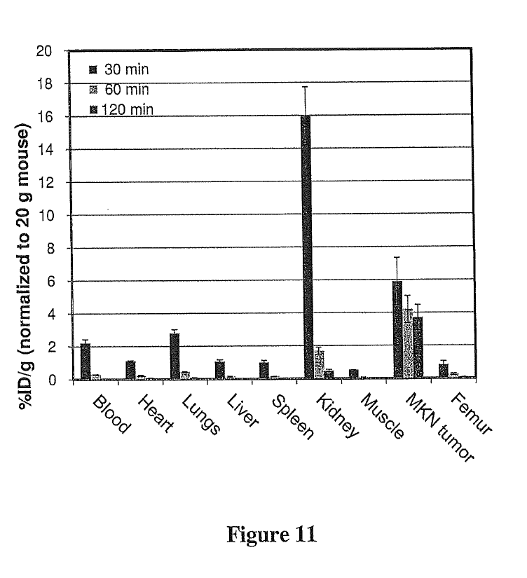

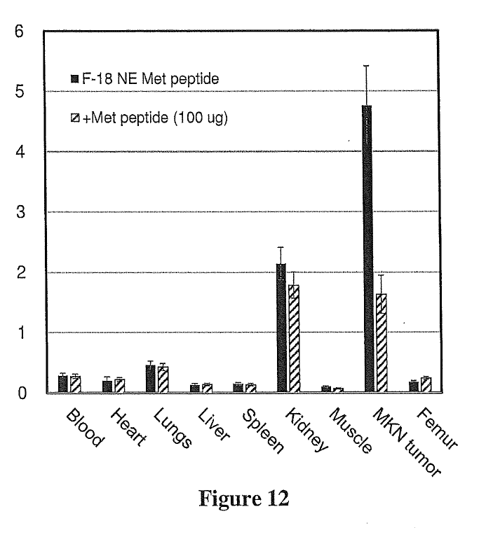

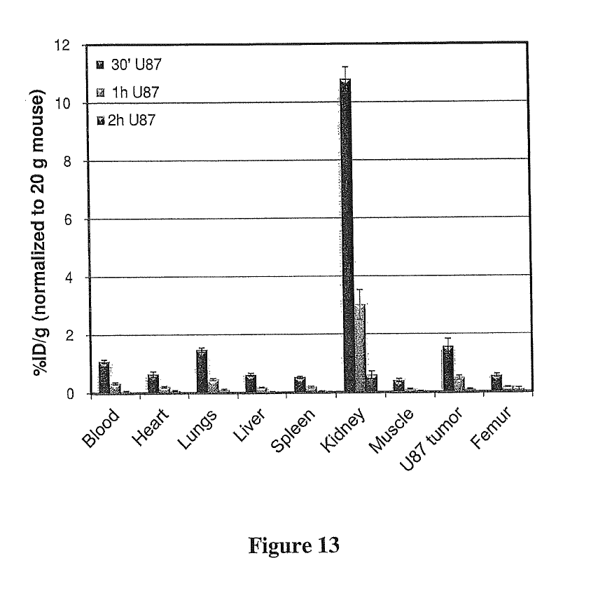

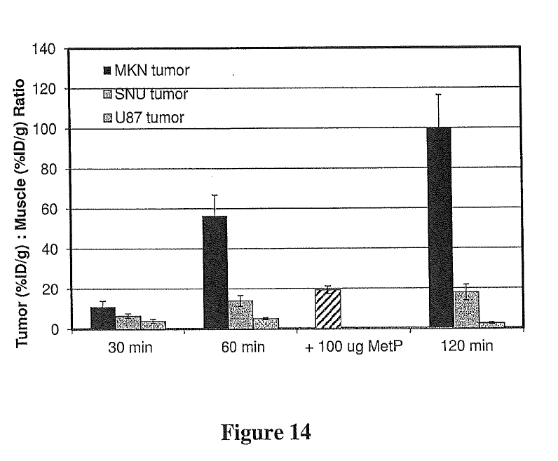

In vivo biodistribution studies were also performed in a xenograft mouse model. Nude mice were injected with MKN-45 cells (gastric carcinoma—high Met levels), SNU-16 cells (gastric carcinoma—moderate Met levels) or U87-MG (glioblastoma—low Met levels), 5-8×10⁶ cells in the flank/shoulder. Blood and tissue uptakes were determined at 30, 60, and 120 min post Compound 1 injections (intravenous) [(% injected dose/g)×body weight/20 (% ID/g; normalized to 20 g mouse)]. The highest uptakes were observed in MKN-45 tumors (6 to 4% ID/g) and kidneys (16 to 0.5% ID/g) at all times. (FIG. 4) Compound 1 was retained in MKN-45 tumors decreasing by approximately 37% from 30 to 120 min whereas in the blood and non-target tissue Compound 1 was quickly cleared from 30 to 120 min with <8% remaining. (FIG. 11) Compound 1 tumor uptake at 60 min was blocked (approximately 60%) in MKN-45 xenografts coinjected with unlabeled Met peptide (MetP, 100 μg) indicating specific binding in vivo. (FIG. 12) With the SNU-16 and U87-MG xenografts, similar uptakes were observed in non-target tissues compared to the MKN-45 xenografts. (FIG. 13, FIG. 14) As expected SNU-16 tumor uptakes (3.5 to 0.64% ID/g) and U87-MG tumor uptakes (1.6 to 0.09% ID/g) were less than MKN-45 tumor uptakes with 2 to 6 fold decreases for SNU-16 tumors and 3 to 40 fold decreases for U87-MG tumors. (FIG. 14) The MKN-45 tumors had the highest tumor:muscle ratios (T:M) of 11:1 (30 min), 56:1 (60 min) and 100:1 (120 min) which increased over time due to clearance of Compound 1 from the muscle [Table 4 (T1)]. MKN-45 T:M ratios obtained from xenografts blocked with unlabeled Met peptide (MetP) were decreased by 65% compared to unblocked (T1). SNU-16 T:M (7:1 to 18:1) and U87-MG T:M (3:1 to 5:1) were decreased from 2 to 60 fold compared to the MKN-45 T:M at the same times (T1).

TABLE 4
¹⁸F-labeled Met peptide Tumor:Muscle Ratios [mean, (SD); n = 4, 5)]

| | Time of Uptake (min) | | | |
|---|---|---|---|---|
| | | | 60* | |
| | 30 | 60 | *(+50 μg MetP) | 120 |
| MKN-45 | 11 | 56 (10) | 19 | 100 (13) |
| (high Met) | (2.8) | | (1.9) | |
| SNU-16 | 6.6 | 14 (2.7) | | 14 (2.1) |
| (moderate Met) | (1.1) | | | |
| U87-MG | 3.8 | 5.0 (0.6) | | 2.5 (0.5) |
| (low Met) | (1.0) | | | |

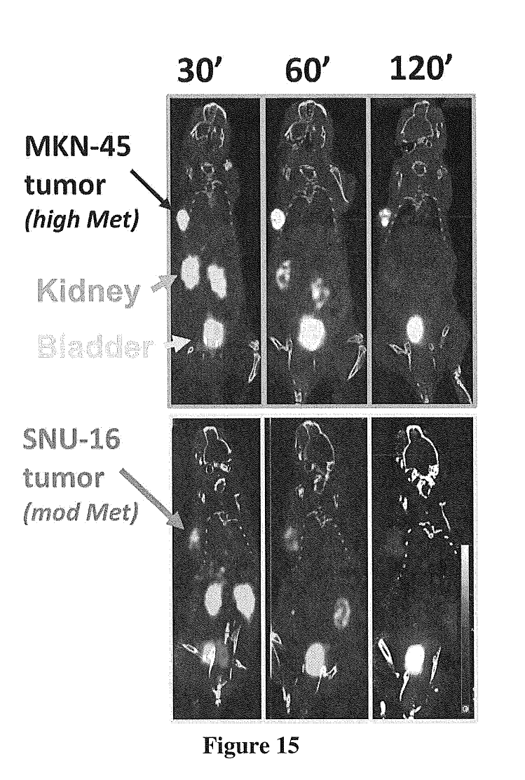

From PET images of MKN xenografts the tumors, kidneys and bladder could be visualized at post-injection imaging times from 30 to 120 min (FIG. 15). Similarly, SNU-16 tumors were discernable in PET images, whereas U87-MG tumors were more difficult to distinguish. Imaging MKN-45, SNU-16 and U87-MG T:M ratios were found comparable to the biodistribution T:M ratios at similar times (data not shown).

Conclusions: Compound 1 exhibited specific and high affinity for Met and had tumor uptakes correlating with Met expression levels in vitro and in vivo. These results suggest that Compound 1 has potential to identify patients whose tumors express moderate to high levels of Met in tumors and therefore, who may benefit from Met-targeted therapies.

### Example 5

### Comparison of Compound 1 and [¹⁸F]AH113804

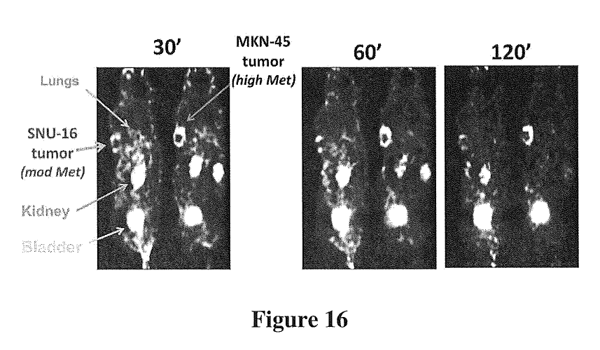

Comparative binding studies performed with [¹⁸F]AH113804 failed to demonstrate specific binding in vitro to high Met expressing tumor cells (MKN-45 and SNU-16). (FIG. 16) It is notable that published results for [¹⁸F]AH113804 do not present in vitro radioligand binding studies for comparison. See, Arulappu et al., c-Met PET Imaging Detects Early-Stage Locoregional Recurrence of Basal-Like Breast Cancer; J. Nucl. Med, 57; pp 765-770 (2016)

The use of the terms “a” and “an” and “the” and similar referents (especially in the context of the following claims) are to be construed to cover both the singular and the plural, unless otherwise indicated herein or clearly contradicted by context. “Or” means “and/or.” The terms first, second etc. as used herein are not meant to denote any particular ordering, but simply for convenience to denote a plurality of, for example, solvents. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An 18-fluorine labeled c-Met peptide comprises Compound 1

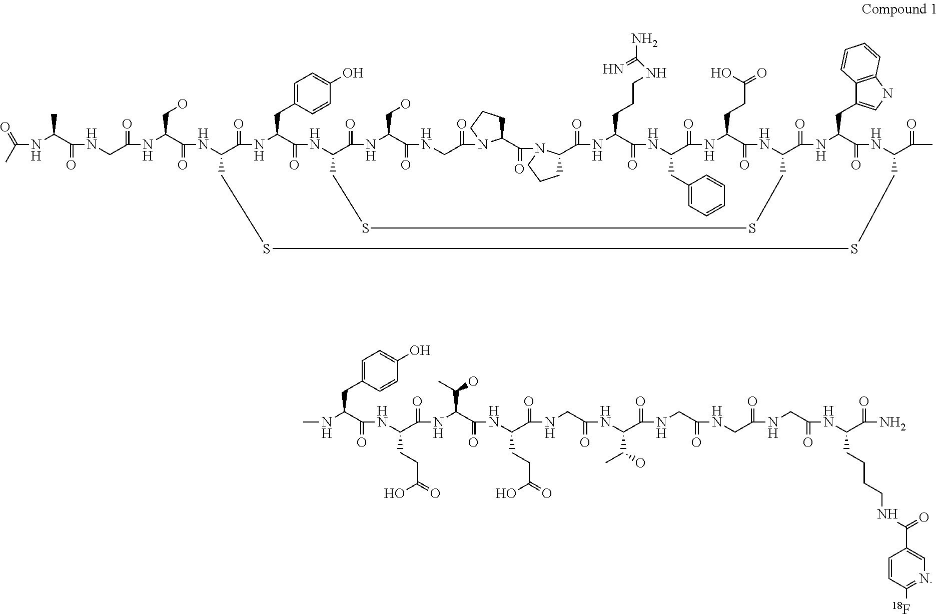

2. A composition comprising the 18-fluorine labeled c-Met peptide of claim 1
and a carrier.

3. The composition of claim 2, wherein the carrier is an aqueous or a non-aqueous carrier.

4. An imaging method, comprising
administering to a subject in need of c-MET imaging a detectable quantity of Compound 1 according to claim 1 and
imaging at least a portion of the subject.

5. The imaging method of claim 4, wherein the imaging is PET imaging.

6. The imaging method of claim 4, wherein the subject in need of c-MET imaging is a subject with a tumor that expresses c-MET, a subject with a tumor that contains c-MET mutations, or a subject that has been treated with a c-MET targeted therapeutic.

7. The imaging method of claim 6, wherein the subject has breast cancer, non-small cell lung carcinoma, glioblastoma, gastric cancer, ovarian cancer, pancreatic cancer, thyroid cancer, head and neck cancers, colon cancer, or kidney cancer.

8. The imaging method of claim 7, wherein the breast cancer is basal-like breast cancer or triple negative breast cancer.

9. The imaging method of claim 7, wherein the subject has glioblastoma or gastric cancer.

10. A base-free method of preparing a fluorine-18 labeled ester of Compound 5, comprising 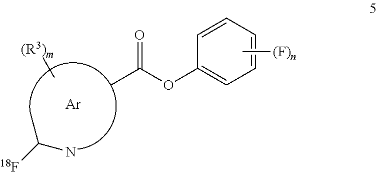
binding [¹⁸F]fluoride to an anion exchange column,
eluting the [¹⁸F] by passing a solution containing Compound 4 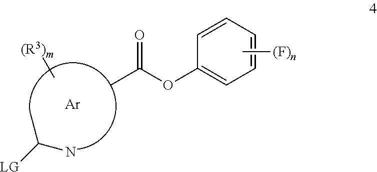
and a solvent through the anion exchange column comprising the [¹⁸F], to provide Compound 5, wherein no base is present during eluting, and wherein
LG is a leaving group, and is —NO₂, —Br, —Cl, —I, or a group of the formula —Y⁺X⁻ wherein Y is —NR¹₃ or —IR² wherein R¹ is a C₁₋₆ hydrocarbyl, and R² is aryl, and X is Br, I, BF₄, O₂CCF₃, ClO₄, OSO₂CF₃, OSO₂C₆H₄CH₃, or OSO₂CH₃,
R³ is NO₂, CN, or F,
the group 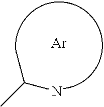
is a C₄₋₇ cyclic aromatic group wherein the bond to the tetra-substituted amine is located on a carbon adjacent to the ring nitrogen,
m is 0 to 3, provided that the valence of the group 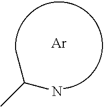
is not exceeded, and
n is 2 to 5.

11. The method of claim 10, wherein Compound 4 is

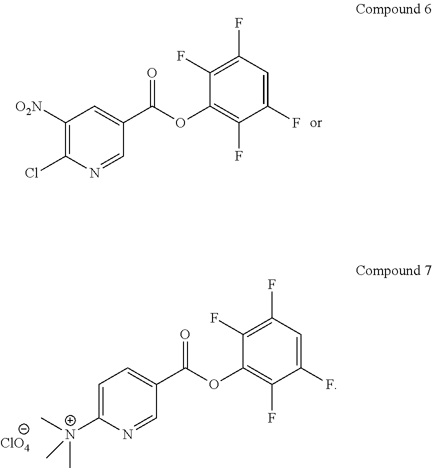 [SMILES: O=C(Oc1c(F)c(F)cc(F)c1F)c1cnc(Cl)c([N+](=O)[O-])c1 ; "ClO4" ; C[N+](C)(C)c1ccc(C(=O)Oc2c(F)c(F)cc(F)c2F)cn1]

12. The method of claim 10, wherein Compound 4 is N,N,N-trimethyl-5-((2,3,5,6-tetrafluorophenoxy)carbonyl)pyridin-2-aminium trifluoromethanesulfonate and Compound 5 is [¹⁸F]fluoronicotinic acid-2,3,5,6-tetrafluorophenyl ester.

13. The method of claim 10, wherein eluting is performed in five minutes or less.

14. The method of claim 10, wherein the solvent comprises acetonitrile, t-butanol, dimethyl sulfoxide, or a combination thereof.

15. The method of claim 10, wherein binding and eluting are performed at room temperature.

16. A method of 18-fluorine labeling a protein, peptide or small molecule comprising a free amine group, the method comprising
binding [¹⁸F]fluoride to an anion exchange column,
eluting the [¹⁸F] by passing a solution containing Compound 4 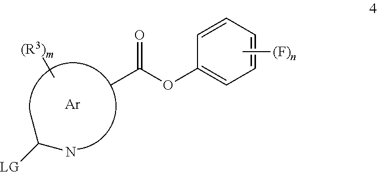
and a first solvent through the anion exchange column comprising the [¹⁸F] to provide Compound 5 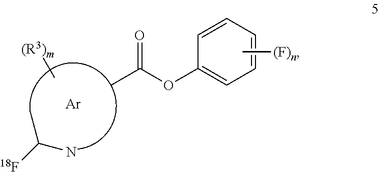
wherein no base is present during eluting; and
reacting the [¹⁸F]fluoronicotinic acid-2,3,5,6-tetrafluorophenyl ester and the protein, peptide, or small molecule comprising a free amine group in the presence of a second solvent and a base to provide the 18-fluorine labeled protein or peptide, wherein
LG is a leaving group, and is —NO₂, —Br, —Cl, —I, or a group of the formula —Y⁺X⁻ wherein Y is —NR¹₃ or —IR² wherein R¹ is a C₁₋₆ hydrocarbyl, and R² is aryl, and X is Br, I, BF₄, O₂CCF₃, ClO₄, OSO₂CF₃, OSO₂C₆H₄CH₃, or OSO₂CH₃,
R³ is NO₂, CN, or F,
the group 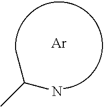
is a C₄₋₇ cyclic aromatic group wherein the bond to the tetra-substituted amine is located on a carbon adjacent to the ring nitrogen,
m is 0 to 3, provided that the valence of the group 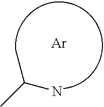
is not exceeded, and
n is 2 to 5.

17. The method of claim 16, wherein Compound 4 is

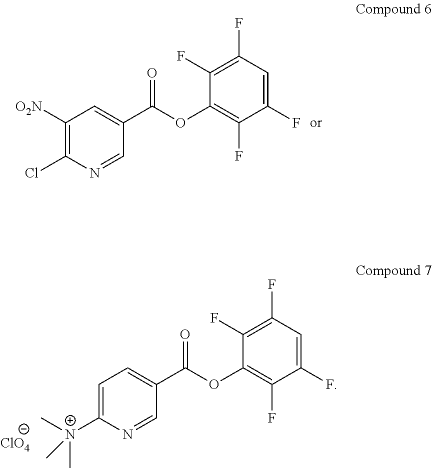 [SMILES: O=C(Oc1c(F)c(F)cc(F)c1F)c1cnc(Cl)c([N+](=O)[O-])c1 ; "ClO4" ; C[N+](C)(C)c1ccc(C(=O)Oc2c(F)c(F)cc(F)c2F)cn1]

18. The method of claim 16, wherein Compound 4 is N,N,N-trimethyl-5-((2,3,5,6-tetrafluorophenoxy)carbonyl)pyridin-2-aminium trifluoromethanesulfonate and Compound 5 is [¹⁸F]fluoronicotinic acid-2,3,5,6-tetrafluorophenyl ester.

19. The method of claim 16, wherein eluting is performed in five minutes or less.

20. The method of claim 16, wherein the first solvent comprises acetonitrile, t-butanol, dimethyl sulfoxide, or a combination thereof.

21. The method of claim 16, wherein binding and eluting are performed at room temperature.

22. The method of claim 16, wherein the second solvent comprises dimethyl formamide, dimethylsulfoxide, acetonitrile, dimethylacetamide, N-methylpyrrolidone, acetonitrile-water, or phosphate buffer.

23. The method of claim 16, wherein the base comprises a secondary or tertiary amine.

24. The method of claim 16, wherein the peptide is a c-Met peptide.
